(19) United States
(12) Patent Application Publication
(10) Pub. No.: US 2009/0208588 A1
(43) Pub. Date: Aug. 20, 2009

(54) GERD carbohydrate compositions

(76) Inventor: Roger Wayne Brown, Phoenix, AZ (US)

Correspondence Address: Roger W. Brown, Ph.D., 810 W. Villa Maria Dr., Phoenix, AZ, 85023, US

(21) Appl. No.: 12/151,394

(22) Filed: May 7, 2008

Related U.S. Application Data
(63) Continuation-in-part of application No. 12/070,313, filed on Feb. 19, 2008.

(51) Int. Cl. A61K 35/64; A61P 1/04

(52) U.S. Cl. 424/537

(57) ABSTRACT

In accordance with the present invention, there is to provide a dietary supplement of essential sugars that provides humans and animals with the essential carbohydrates needed to maintain proper health and to provide a healthier alternative to PPI medications for the treatment of GERD using a mixture of honey and essential sugar carbohydrates and to provide pets with an equal level of medications as that afforded to humans.

### RELATED APPLICATIONS

This application claims priority on patent application Ser. No. 12/079,907 filed with the US PTO on Mar. 31, 2008 and on patent application Ser. No. 12/070,313 filed Feb. 19, 2008, and is a continuation-in-part of patent application Ser. No. 12/070,313 filed Feb. 19, 2008, the entire disclosure of which is incorporated herein.

The present application is related to U.S. Pat. No. 7,358,245 B2, issued Apr. 15, 2008, included by reference herein.

The present application is related to U.S. Pat. No. 7,202,220 B2, issued Apr. 10, 2007, included by reference herein.

The present application is related to U.S. Pat. No. 7,199,104 B2, issued Apr. 3, 2007, included by reference herein.

The present application is related to U.S. Pat. No. 7,196,064 B2, issued Mar. 27, 2007, included by reference herein.

The present application is related to U.S. Pat. No. 7,157,431 B2, issued Jan. 2, 2007, included by reference herein.

The present application is related to U.S. Pat. No. 6,929,807 B1, issued Aug. 16, 2005, included by reference herein.

The present application is related to U.S. Pat. No. 3,890,438 A, issued Jun. 1, 1975, included by reference herein.

The present application is related to U.S. Pat. No. 3,947,601 A, issued Mar. 1, 1976, included by reference herein.

The present application is related to U.S. Pat. No. 4,260,603 A, issued Apr. 1, 1981, included by reference herein.

The present application is related to U.S. Pat. No. 4,466,958 A, issued Aug. 1, 1984, included by reference herein.

The present application is related to U.S. Pat. No. 4,777,045 A, issued Oct. 1, 1988, included by reference herein.

The present application is related to U.S. Pat. No. 4,871,557 A, issued Oct. 1, 1989, included by reference herein.

The present application is related to U.S. Pat. No. 5,612,039 A, issued Mar. 1, 1997, included by reference herein.

The present application is related to U.S. Pat. No. 5,827,526, issued Oct. 1, 1998, included by reference herein.

The present application is related to U.S. Pat. No. 7,244,706, issued Jul. 17, 2007, included by reference herein.

The present application is related to U.S. Pat. No. 7,323,179, issued Jan. 29, 2008, included by reference herein.

The present application is related to U.S. patent application Ser. No. 12/070,313 filed with the US PTO on Feb. 19, 2008, for Compositions of Carbohydrates as Dietary Supplements, by Roger W. Brown, Ph.D.

The present application is related to U.S. patent application Ser. No. 12/079,907 filed with the US PTO on Mar. 31, 2008, for Compositions of Carbohydrates as Dietary Supplements, by Roger W. Brown, Ph.D.

US Class: 514/8; 514/230.2; 514/23; 514/54; 514/62; 424/725

### FIELD OF THE INVENTION

The present invention relates to the field of dietary supplements for mammals promoting good nutritional health and, more particularly, to the compositions of carbohydrates as dietary supplements used to treat the condition known as GERD.

### BACKGROUND OF THE INVENTION

Gastroesophageal reflux disease, GERD, is the most prevalent upper gastrointestinal tract disease known today. Current pharmacotherapy aims at reducing gastric acid secretion, or at neutralizing acid in the esophagus as a treatment. The major mechanism behind reflux has been considered to depend on a hypotonic lower esophageal sphincter. However, e.g. Holloway & Dent, 1990, Gastroenterol. Clin. N. Amer. 19, pp. 517-535, has shown that most reflux episodes occur during transient lower esophageal sphincter relaxations, TLESRs, i.e. relaxations not triggered by swallows. It has also been shown that gastric acid secretion usually is normal in patients with GERD.

GERD is caused by reflux of gastric contents into the esophagus leading to heartburn and other typical symptoms. In many cases, an inflammation develops in the distal esophagus, esophagitis. It has been known for a long time that suppression of production of gastric acid ameliorates both symptoms and esophagitis. However, some patients continue to have symptoms despite adequate control of acid secretion. Reflux of other noxious factors is believed to be responsible for those symptoms. Most focus has been centered on the importance of bile acids, and the development of severe GERD is related to the degree of esophageal bile acid exposure.

Mammal's bodies produce a large number of different types of chemicals that the body uses to ward off disease, retard cell degradation, maintain memory and maintain overall body health. These chemicals are produced as a byproduct of what the mammal has eaten. If all of the right foods are eaten in the proper amounts then the body will produce enough of all of the chemicals required to keep it functioning properly. Over the years people have sought after which chemicals are actually necessary for good health and which ones are just good. As this field is evolving more and more information is being discovered about what chemicals mammal's bodies require for proper functionality.

Over the last ten years a lot of research has been done concerning cell communications and its importance to a properly functioning mammal's body. This research indicates that there are eight essential sugars that all mammals need in order to stay healthy; http://www.glyconutrients-center.org/ and http://www.glyconutrientsreference.com/. Six of these carbohydrates (sugars) are generally missing in the diets of most humans and seven are missing from the diet of most animals. However, very small concentrations of these missing carbohydrates are contained in various plants and some sea products. A synopsis of seven of these essential sugars/carbohydrates and what functions they have been found to influence follows:

D-Galactose is readily available in human diets but not in most animal diets. It is obtained from the conversion of lactose (milk sugar) and is also easily obtained from dairy products unless you suffer from lactose intolerance or are a vegetarian who does not eat dairy products.

D-Mannose is not readily available in our diets. The most popular source is Aloe Vera but it is also readily available from a number of other sources. It is also available in tiny quantities in the bran of whole wheat and a few other cereal crops. However, it is very unstable in its pure form and must be taken fresh from the plant and properly standardized to be of any benefit. It plays a profound role in cellular interactions and has even been known to lower blood sugar levels. It is absolutely vital to proper immune defenses against microbial invaders and has a natural and powerful anti-inflammatory effect. This sugar is readily available in supplemental form. Good for: Wound healing, Diabetics, Anti-viral, Anti-inflammatory and Arthritis.

N-Acetyl-Glucosamine is not readily available in our diets. It is particularly beneficial for cartilage regeneration and joint inflammation. Glucosamine derivatives are well-known natural medicine for arthritic conditions comes from this sugar compound. It has many more therapeutic effects and deficiencies or malfunctions of this sugar have been linked to diseases of the bowel. Derivatives of this sugar are readily available in supplemental form. Good for: Wound repair, Range of motion, Insulin production, Arthritic conditions, Learning, HIV and Vision.

L-Fucose is not readily available in our diets but is readily found in breast milk, astragalus herb, in several medicinal mushrooms, and in certain brown algae. It has numerous well-documented benefits for the immune system and has been shown to inhibit some cancer growth and metastasis. Good for: Long term memory, Anti-viral, Cancer and tumors and Skin allergies.

D-Xylose is not readily available in our diets. It is often seen in sugarless gums, candies, etc. in that it has a sweet taste but does not cause tooth decay. It has recently been added to nasal sprays and appears to discourage the binding of allergens and pathogens to mucous membranes. It also has anti-bacterial and anti-fungal properties and may help prevent certain cancers. Good for: Anti-fungal and gram negative bacteria.

N-Acetyl-Neuraminic Acid is not readily available in our diets but is another sugar that abounds in breast milk and dramatically impacts brain function and growth. It, too, boosts immune function and has documented anti-viral actions. Interestingly, in certain disease states the ability to digest this sugar is impaired. Good for: Anti-viral, Kidney stones, Asthma, Learning and Arthritis.

N-Acetyl-Galactosamine is not readily available in our diets. It is the least known of the essential sugars although it appears to inhibit the growth of some tumors and, like the other sugars, plays an individual role in keeping cellular messages clear and promptly delivered. Most of these sugars do not involve or require insulin for their use and go directly to the cells where they are incorporated into the cell structure wherever they are needed. Good for: Heart disease, Aging (cell rejuvenation), Joint functioning and Vision.

The actual body requirements for these missing carbohydrates has been hard to estimate because of their rarity and because of this the FDA has not set a lower daily intake limit on any of them. However, research has indicated a level of dose for each of these carbohydrates required to produce noticeable effects. The base level for each of these carbohydrates is about 0.005 mg/kg of body weight or 0.4 mg/day for a 150 pound mammal. These levels correspond to base levels of other medications.

The base level is not the required level to start seeing results but the level below which nothing much has been seen. The general therapeutic levels are above 0.1 mg/kg of body weight or 8 mg for a 150 pound mammal. These findings indicate that the minimum required level for these essential carbohydrates is a hundred times larger than is available in natural foods including the specially prepared supplements designed to alleviate these missing carbohydrate deficiencies.

The seven essential sugars/carbohydrates are: D-Galactose, L-Fucose, D-Mannose, D-Xylose, N-Acetyl-Glucosamine, N-Acetyl-Neuraminic Acid, and N-Acetyl-Galactosamine. Of these seven carbohydrates D-Mannose and Glucosamine derivatives are available as full strength supplements from a large number of over the counter drug stores and D-Galactose is available from specialty suppliers. The remaining four carbohydrates are much too expensive for companies to currently package in full strength so all that is generally available to the public are extremely low concentrations, trace amounts, in food substitutes.

While there are several different companies selling glyconutrients only one of them has filed for patent protection in the US. This company is Mannatech, Inc. of Coppell Tex. and they sell their glyconutrients through a chain of 500,000 independent dealers worldwide. They filed an initial US patent application in 1997 and was granted a US patent on Aug. 16, 2005; U.S. Pat. No. 6,929,807 B1. Since then they have filed four additional filings to this one patent. They were granted additional patent numbers which are: U.S. Pat. No. 7,157,431 B2, U.S. Pat. No. 7,196,064 B2, U.S. Pat. No. 7,199,104 B2, and U.S. Pat. No. 7,202,220 B2.

Their first patent sets out eight essential sugars and ties these sugars to food sources where they can be found. However, they don't disclose the actual amount of the various essential sugars/carbohydrates in these food sources. Their web site is located at: https://www.mannatech.com/Default.aspx

They produce specific supplements used to address several different conditions that arise in mammals: weight management, alcoholism, nutrition, wellness management, lifestyle management, growth essentials, performance management, skin care and performance nutrition, but they don't include GERD. All of their products are different mixes of the same basic foods, as seen above, and as can be seen they contain very little of what is required by the body to function properly.

Another company also selling glyconutrients is shown here: http://www.naturalcureguide.com/glyconutrients.html Again, as Mannatech does, this company also provides the greatest amounts of the carbohydrates that are not in short supply in the body and not much else.

While all of the companies selling glyconutrients have products specifically orientated to correct certain illnesses none of them have anything that addresses the common cold, cold remedies, Group 1 or Group 5 viruses, shingles, influenza, cold sores, oral ulcers, memory or cell aging, animal supplements, or GERD. Clearly these missing carbohydrates have capabilities in all these areas but these areas are not being addressed by any of the current glyconutrient companies.

One of the short comings with the current offerings to the public is the lack of these missing carbohydrates at therapeutic dose levels (levels at which changes are seen in hours instead of many months or years). For example, L-Fucose is available in Gum Tragacanth ($34/pound) and Brewer's Yeast (half a pound for $6), but the availability of L-Fucose in Gum Tragacanth is only 0.1% by weight and only 0.05% in Brewer's Yeast. Currently available glyconutrient supplements supply less than 0.1 mg of L-Fucose per daily dose.

N-Acetyl-Galactosamine is available in shark cartilage (3 oz. $16) but there is only 0.01% by weight of it there. By taking the specially made glyconutrient supplements available from the above companies the daily dose of N-Acetyl-Galactosamine is still generally under 0.1 mg. Likewise, N-Acetyl-Neuraminic Acid is available in Whey Protein (36 26 gram servings for $33) and Hen's eggs but there is only 0.02% of it there by weight. A daily dose from one of the special glyconutrient supplements generally has less than 0.2 mg of it available.

Mannatech even admits that their formulations are extremely weak and require many months to see any results at all “Be patient! Research shows that it may take up to 4 months (or more) to notice the effects of any changes you make to your diet.” https://www.mannatech.com/Shopping/Product.aspx and also see https://www.mannatech.com/Shopping/RDReports.aspx

There have been numerous complaints that Manntech products do not provide any benefit at all even after months of usage; http://www.reviewcentre.com/reviews274152.html “I saw Mannatech Ambrotose, Glyconutrients advertised on a Fibromyalgia site. I was contacted by a Mannatech Associate and was advised that Ambrotose could help with all my ailments—Fibromyalgia, Osteoarthritis, Asthma, allergies etc. I was also told that I wouldn't need to continue to take the vitamin, mineral and herbal supplements I had been using. Now, 4 months on, my asthma has worsened, my cholesterol levels have gone up and my fibromyalgia and arthritis are unchanged.”

From this it can be seen the currently available special glyconutrient supplements sold to replace these missing carbohydrates contain mainly filler material and other chemicals that are already available to the body through other sources or are not needed by the body at all. Additionally, many of these special supplements contain ingredients known to excite an allergic reaction in a large part of the population; such as Whey Powder and Aloe Vera ingredients. On the other hand there is no known allergic reaction to these essential sugars when taken in their pure form even at therapeutic dose levels.

One of the essential sugars (a carbohydrate), N-Acetyl-Neuraminic Acid (Sialic Acid), has been shown to be more than 1000 times more effective at killing viruses than any other known medication when used in therapeutic doses “Another study reported in a 1995 issue of Antimicrobial Agents and Chemotherapy, stated that a sialic acid mixture was up to 1000 times more effective in fighting influenza than potent antiviral drugs. Such viruses can also cause cold sores, hepatitis, viral pneumonia, as well as the common cold.” http://www.glyconutrients-center.org/N-acetylneuraminic-acid.php

Even though the effectiveness of Sialic Acid as an antiviral agent been known for over a decade it is still waiting to be offered to the public in therapeutic dose levels.

The treatment of gastroesophageal reflux disease, GERD, has been very difficult prior to the recent introduction of Proton Pump inhibitors, PPI, by several pharmaceutical companies. The FDA has approved use of these medications for short term treatment. However, the long term effects of taking these medications is not known. What is known is that they produce residue that are not dissolved, excreted or used as a food source. As such these residues are neither filtered or excreted from the body by either the kidney or the liver and just accumulate in the body with unknown results.

In late 2007 the FDA issued two news releases regarding PPI medications on their website: http://www.fda.gov/Medwatch/SAFETY/2007/safety07.htm
[UPDATE Dec. 11, 2007] FDA informed healthcare professionals of the issuance of the Agency's follow-up communication regarding its review of safety data for the drugs omeprazole and esomeprazole that raised concerns about a potential increased risk of heart problems for patients treated with these drugs. The Agency conducted a comprehensive review of the data from two studies that were submitted to FDA. FDA continues to believe that long-term use of omeprazole or esomeprazole is not likely to be associated with an increased risk of heart problems and recommends that healthcare providers continue to prescribe and patients continue to use these products in the manner described in the labeling for the two products. See the “Update of Safety Review” for information regarding the two studies that were reviewed.
[Posted Aug. 9, 2007] FDA issued an early communication about the ongoing review of new safety data for the proton pump inhibitors, Prilosec and Nexium. The new safety data was from two small long-term clinical studies in patients with severe gastroesophageal reflux disease (GERD). In both studies, patients were randomly assigned to receive treatment with a drug (either omeprazole or esomeprazole) or to have surgery to control their GERD.
The results from the study of Prilosec and analyses from an ongoing study of Nexium raised concerns that long-term use of Prilosec or Nexium may have increased the risk of heart attacks, heart failure, and heart-related sudden death in those patients taking either one of the drugs compared to patients who received surgery. After reviewing these and other data submitted by the company, FDA's preliminary conclusion at this time, is that collectively, these data do not suggest an increased risk of heart problems for patients treated with omeprazole or esomeprazole. Healthcare providers should not change their prescribing practices and patients should not change their use of these products at this time.

Outside of medical diagnostic manuals, GERD is frequently described in direct-to-consumer advertising as “heartburn” caused by “excess stomach acid”. This lack of continuity in the definition of GERD is troubling because it misinforms people about the true cause of GERD and opens the door for large scale, unnecessary prescribing of PPIs. Diagnosing GERD as an excess of stomach acid ignores important underlying causes of heartburn and indigestion: overeating of greasy and spicy foods, processed foods, eating on the run without thoroughly chewing food, lying down soon after eating and continually eating foods that trigger heartburn. Stomach acid production is a normal physiological response to these habits and conditions.

A less known problem with the use of PPIs is that getting off them can present a big obstacle. When a person stops taking PPIs there can be a large surge in the production of stomach acid accompanied by increased heartburn, indigestion and reflux. Using PPIs long term can lead to a vicious and biologically abnormal acid cycle.

According to Jonathan V. Wright, MD, founder of the Tahoma Clinic in Washington and author of “Why Stomach Acid is Good for You” GERD is actually a rare disorder. However, as the drugs developed to shut down stomach acid have become some of the best selling drugs in the history of pharmaceuticals, GERDS diagnosis has increased. The FDA list of adverse reactions to Nexium could lead to a sharp increase in the number of other medications just used to treat adverse reactions to Nexium in a patient. The risk of taking PPI with children is even more problematic.

Proton pump drugs very effectively shut down the production of stomach acid by specialized cells in the stomach. Dr. Wright says that proton pump inhibitors like Nexium, Prevacid and Prilosec are “the most potent of the acid suppressing drugs and that just one Nexium is capable of decreasing stomach acid by 90-95% for most of the day”.

Shutting down stomach acid production is a very severe treatment that puts the healthy growth and development of children at risk. Turning off 90-95% of a child's normal production of stomach acid increases their risks for:
1. Food borne pathogenic infections
2. Decreased digestion and absorption of protein and protein malnutrition
3. Impaired development of bone tissue, skin cells, ligaments, tendons, gum tissue, arterial cells which depend on the normal production of the protein collagen for structural integrity
4. Impaired synthesis of biological proteins including enzymes and hormones. The gastric enzyme pepsin is vital to protein digestion. The hormones insulin and glucagon regulate blood sugar, thyroxin regulates the body's metabolic rate, calcitonin and parathyroid hormone regulation, bone mineralization, and antidiuretic hormone regulates fluid electrolyte balance (the vital acid/base balance of cellular mineral salts including calcium, magnesium, sodium, potassium, chloride, phosphorus, bicarbonate, sulfates, protein and organic acids)
5. Food allergies and leaky gut syndrome caused by incomplete protein digestion
6. Calcium deficiency accompanied by abnormal bone mineralization
7. Iron deficiency with risk for anemia
8. Zinc deficiency with risk for impaired immune function
9. Folate deficiency with risk for large cell anemia, elevated homocysteine levels and neurological dysfunction
10. Vitamin B12 deficiency with risk for pernicious anemia, large cell anemia and neurological dysfunction and asthma in children

This is an abbreviated list of the health risks of shutting down stomach acid production in children as well as in adults. It illustrates the role that normal stomach acid plays in the absorption and digestion of nutrients, its role in protecting us from food borne pathogens that are on the rise in our food supply.

Even though patients take PPI medications to relieve them of the reflux symptoms of GERD most of them are still plagued with reflux. “Most patients with gastroesophageal reflux disease continue to have reflux despite twice-daily dosing with a proton pump inhibitor, according to results of a study reported here. The study involved 167 patients who underwent MII-pH studies while on twice-daily PPI therapy. Drugs represented in the study were esomeprazole (Nexium), omeprazole (Prilosec and others), lansoprazole (Prevacid), rabeprazole (Aciphex), and pantoprazole (Protonix). The mean number of non-acid reflux episodes during 24-monitoring ranged from 34 to 37 across the different drugs, and the number of acid episodes averaged five to 10 per 24 hours (P<0.05 versus non-acid reflux).”

“Using the criterion of 48 reflux episodes to define abnormal reflux, Dr. Castell found that 22% to 50% of patients had abnormal findings, and the proportion of patients with abnormal reflux did not differ significantly among the PPIs. Reflux continued to occur, and it didn't make any difference which PPI a patient was taking,” said Dr. Castell.” http://www.medpagetoday.com/MeetingCoverage/ACG/tb/6991

A search on the internet yielded dozens of web sites that offered cures for GERD. Most of these sites either sell their secret recipe or propose taking an anti-acid type of medication. An anti-acid type of medication would be anything that contained calcium carbonate in it. A number of these ‘Cure Your GERD’ web sites proposed various other compositions to cure GERD. Most of these other compositions contained vinegar. A few of these web sites also added bee honey to their vinegar mixtures as a sweetening ingredient so the patient could get the vinegar down without throwing up. A very few sites also propose using various forms of honey from artificial honey to natural bee honey to cure GERD.

However, a lot of sufferers that tried these honey remedies had extremely poor results at suppressing GERD symptoms. Even those that used honey alone did not achieve the expected results. “Several sites on the internet claim that bee honey is a cure for heartburn. Is it? It never actually worked for me.”, www.manageyourheartburn.com/blog/honey-does-it-help/

“Molasses also seems to work for me but honey makes me worse even the organic honey”, www.medhelp.org/forums/gastro/messages/37321.html

Similar to the individuals experiences mentioned above we found using any available honey generally didn't alleviate GERD reliably. However, when we used a specially prepared type of honey and added a mixture of essential sugars to this honey the new composition produced very predictable and reliable results for treating GERD.

During our testing we found that clover honey that was filtered and processed at a temperature between 100 degrees Fahrenheit, F, and 140 degrees F. worked as a treatment for GERD. Other types of honeys and honey processed in other ways only worked in a random hit and miss fashion at treating GERD at best.

When using this specially prepared honey alone we found the results were still not all that predictable or consistent in treating GERD. However, when we added a composition of essential sugars to this honey the results became very consistent and reliable in treating GERD.

Honey is primarily a composition of carbohydrates. These carbohydrates are all derivates of the essential sugars. Honey contains four main sugars mixed in 17% water; Levulose (D-Fructose) 38%, Dextrose 31%, Maltose 7% and Sucrose 1%. The remaining 6% is comprised of vitamins and minerals. Honey contains more Ascorbic Acid than any other vitamin and more Potassium than any other mineral, Calcium being second and Magnesium is third. There are also trace amounts of enzymes and other chemicals in bee honey. These trace chemicals are generally local to the area where the honey is gathered by the bees. Attempts to produce bee honey artificially has not been very successful. While the artificial honey looks and tastes like real honey it seems to be missing something, for when this artificial honey is fed to bees they die.

It is therefore an object of the invention to aid the body's ability to fight colds and infections by providing it with the correct mix and level of carbohydrates needed by the body to fend off the infection.

It is another object of the invention to aid the body's ability to lessen the cells aging process by providing it with the correct mix and level of carbohydrates needed by the cells to keep them healthy.

It is another object of the invention to give an animal the ability to fight viruses and infections much more effectively by providing it with the correct mix and level of carbohydrates needed by its body.

It is another object of the invention to promote good health and wellness to all types of mammals through the proper mix of carbohydrates targeted to specific aliments, like increasing mental ability and learning, wound repair and long term memory.

It is another object of the invention to promote good health by offering a safe and healthy alternative to using PPI medications for the treatment of GERD without incurring any of the side effects and negative long term effects prevalent in the current PPI medications.

It is another object of the invention to provide a method for the treatment of gastroesophageal reflux disease, GERD, whereby a pharmaceutically and pharmacologically effective amount of a honey and an essential sugar composition is administered to a subject in need of such treatment.

It is another object of the invention to provide a method for the treatment or prevention of regurgitation, whereby a pharmaceutically and pharmacologically effective amount of a honey and an essential sugar composition is administered to a subject in need of such treatment.

### SUMMARY OF THE INVENTION

In accordance with the present invention, there is to provide a dietary supplement that provides a mammal with the essential carbohydrates needed to maintain proper health and functionality, to fend off illness, provide viral defenses, lessen the aging process of cells, to provide a healthier alternative treatment to PPI medications for the treatment of GERD and to provide pets with an equal level of medications as that afforded to humans.

### BRIEF DESCRIPTION OF THE DRAWINGS

There are no drawings.

### DESCRIPTION OF THE PREFERRED EMBODIMENT

The carbohydrates, including honey, included in the dietary supplement of the invention are available from a number of manufactures. Most are derived synthetically from other pure chemicals rather than being expensive plant or animal derivatives. A supplier for the two most expensive essential sugars, Sialic Acid (CAS# 131-48-6) and N-Acetyl-Galactosamine (CAS# 1811-31-0), is R&S PharmChem located in China http://www.rspharmchem.com. A supplier for L-Fucose (CAS#2438-80-4) is AppliChem located in Germany http://www.applichem.de/perl/catalog/catalog.pl. AppliChem can also supply two other more readily available essential sugars, D-Galactose (CAS#59-23-4) and D-Xylose (CAS#58-86-6). D-Mannose (CAS#3458-28-4) is available from a number of the larger supplement suppliers like NOW Foods http://www.nowfoods.com/. The remaining carbohydrate, N-Acetyl-Glucosamine (CAS#98632-70-3) is generally used in one of its many derivative forms. This invention uses the derivatives Glucosamine HCL (CAS#66573-21-5) and Glucosamine Sulfate (CAS#29031-19-4) instead of Glucosamine (CAS#3416-24-8). Both of these carbohydrates are readily available at drug stores. It should be recognized that the composition of the carbohydrate is not intended to be limited by the source from which it is obtained.

It should be stressed that this invention does not incorporate the use of Glucose or Acetylated Mannose. While Glucose is one of the eight essential sugars it is so prevalent in today's diets that adding additional amounts of Glucose in a supplement generally provides no useful benefit. Acetylated Mannose is a plant derivative from the Aloe Vera plant obtained during a special purification process of Mannose from Aloe Vera residues. Acetylated Mannose has not been shown by independent research to be of any beneficial use as a dietary supplement.

Although the present invention includes the above cited seven essential sugars and honey (carbohydrates), it should be noted that other carbohydrates, nutritional compounds or biologically active or inert compounds can be included in the dietary supplement of the invention. Such other ingredients may include spices, scents, thickening agents, emulsifiers, fragrances, flavorings, buffers, gels, binders, filler material, lubrication material, vitamins and or minerals and/or other such compounds that facilitate the formulation or administration of the inventive dietary supplement. These components can be provided separately to a mammal given said dietary supplement.

Many different types of vitamins and minerals can be included in the dietary supplement of the invention. While a few vitamins and minerals of synthetic origin do possess nutritional value, particular embodiments of the dietary supplement herein can contain nutritionally effective amounts of non-toxic vitamins and minerals obtained predominantly from natural sources.

Other compounds, agents and nutrients can also be included in the dietary supplement of the invention, for example: cellulose, calcium carbonate, stearic acid, amino acids, glycine, glycerin, glycerine, olive oil, essential fibers, essential oils, essential botanicals, essential enteric ecology, flora growth promoters, emulsifiers, essential fatty acids, fungi, fungal extracts, plant extracts, scents, fragrances, and enzymes.

As all of seven of these essential sugars have a molecular mass well under 600 they can all be readily absorbed through the skin. This property allows these sugars to target areas that are very near the surface of the skin that would be difficult or impossible to reach by other means. To this end they could be made into pastes, spreads, jelly, sweeteners, saves and lotions and rubbed on the areas of the body where a particular surface or subsurface effect was desired.

Independent research indicates that these essential sugars are not stored in the body. After ingestion the sugars are assimilated into the blood stream within minutes (if taken orally on an empty stomach). Once in the blood stream they flow through the body and cells in need of these nutrients take what they need and the rest flows on. Most of these unused sugars are excreted via the urine within 12 hours after ingestion. The tests indicate that while excess of these sugars are excreted from the body within 12 hours the cells maintain an internal supply of these sugars for a period of up to a week. Studies have also shown that taking these seven essential sugars, in the levels covered by this invention, did not cause an abnormal rise in the blood sugar levels of diabetics.

The dietary supplement form of the invention has been prepared and human testing of it began in 2006 and animal testing began in 2007. It can be administered to mammals in powdered form, reconstitutable powder, ingested powder, liquid-solid suspension, liquid, nasal spray, inhalation, hypodermic injection, skin patch, subcutaneous injection, paste forms, suave, capsule and tablet dosage forms. It should be readily obvious to one of ordinary skill in the science of formulations that the present dietary supplement can also be formulated appropriately for irrigation, ophthalmic, rectal, sublingual, transdermal buccal, vaginal, or dermal administration. Thus, other dosage forms such as chewable candy bar, concentrate, drops, elixir, emulsion, film, gel, granule, chewing gum, jelly, lotions, oil, paste, pastille, pellet, suave, shampoo, rinse, soap, sponge, suppository, swab, syrup, chewable gelatin form, or chewable tablet can be used.

Due to varying diets among people, the dietary supplement of the invention can be administered in a wide range of dosages and formulated in a wide range of dosage unit strengths. For example, for those people who are missing from their diet seven of these essential carbohydrates, a dietary supplement containing those carbohydrates in nutritionally effective amounts can be formulated. As well, for those people whose bioabsorption of essential carbohydrates is extremely efficient, a dietary supplement formulation containing reduced amounts of essential carbohydrates can be prepared.

It should be noted that the dosage of the dietary supplement can also vary according to a particular ailment or disorder that a mammal is suffering from when taking the supplement. For example, a person suffering from chronic colds will generally require a dose different than an animal would who is sick in order to obtain a benefit. An appropriate dose of the dietary supplement can be readily determined by monitoring patient response, i.e., general health, to particular doses of the supplement. As well, when another agent such as a vitamin and/or a herbal extract is being administered to a mammal along with the present carbohydrate dietary supplement, the appropriate doses of the supplement and each of the agents can be readily determined in a like fashion by monitoring patient response, i.e. general health, to particular doses of each.

It is contemplated by the invention that the dietary supplement can be administered simultaneously or sequentially in one or a combination of dosage forms. While it is possible and even likely that the present dietary supplement will provide an immediate overall health benefit, such benefit may take hours or days to materialize. Nonetheless, the present carbohydrate dietary supplement will provide a beneficial nutritional response in a mammal consuming it.

For the examples herein, the dietary supplement of the invention was administered as a powder-containing capsule. According to the capsule size and ingredients used in a given study exemplified herein, the dietary supplement was administered by oral ingestion. The indicated doses for humans in Example 1 are based upon #00 sized capsules.

### Example 1

A suitable composition for a product according to the present invention is shown in the following table.

| | Weight % | Weight % | Human |
|---|---|---|---|
| Carbohydrate | (range) | (tested) | (mg) |
| D-Galactose | 0.1 to 40 | 6.3 | 43 |
| L-Fucose | 0.1 to 90 | 3.1 | 21 |
| D-Mannose | 10 to 70 | 52.1 | 358 |
| D-Xylose | 0.1 to 70 | 6.3 | 43 |
| Glucosamine HCL | 10 to 60 | 25.1 | 173 |
| Sialic Acid | 0.1 to 50 | 6.5 | 45 |
| N-Acetyl-Galactosamine | 0.1 to 90 | 0.6 | 4 |

In this composition the ingredients Glucosamine HCL, D-Galactose and D-Mannose are optional and preferred. Instead of using Rice Flour as an inactive filler as is done in most products currently available to the public the three optional ingredients set out above were used as active fillers. A disadvantage of using Rice Flour as an inactive filler is its high glycemic index tends to drive a mammal's Triglyceride levels up very high, while using essential sugars as fillers doesn't.

The ingredients are typically in a powered form and are dry blended in a mixer. The mixture can then be packaged as a blended powder into capsules or caplets. In this example the mixture was packaged into size 00 capsules with an average weight of 687 mg for human doses and 25 mg for animal doses. The mg per ingredient for animals would be found by dividing the human ingredient dose in mg by 27.5, for example: for D-Mannose the animal ingredient dose would be 13.0 mg.

This composition is considered a health maintenance mix. It was designed to reduce the probability of infections, like colds, while taking the composition. There were three test trials run using this composition on both humans and animals. These three tests ran from late January 2007 to late December 2007. In the first test, that lasted for two weeks, a capsule/dose was administered twice a day for two weeks. The next test lasted for one month during which time one capsule/dose was administered per day at bedtime.

The third test lasted slightly over nine months and the dose was one capsule/dose a week administered at bedtime. During that time there were no deaths or adverse reactions to the composition by anyone in the test group either human or animal. Regular blood, lipid and electrolyte testing was done. A base line was run prior to the test and during the test blood testing was done regularly to determine if there were any adverse effects due to taking the composition.

The end result was that no one taking this composition contracted a cold or any other type of viral infection during the entire period of the test, even though one of the individuals in the test was prone to chronic colds and flu. This test extended through two different flu seasons and the test subjects continued to work everyday and come in contact with infected people on a daily basis yet they didn't catch anything while taking this composition. The blood tests didn't show any adverse effects from taking this composition.

In a follow up test these capsules were taken on a once a month basis by the same human test group from the first test, this time during the flu season. Within two weeks following a single monthly dose (this was during the four week pause after the end of the weekly tests) two of the subjects came down with a cold or flu. After this the test was stopped and no additional capsules were taken and within two weeks all the test subjects resumed their normal activity with no adverse or lasting effects.

During the animal tests old cats were exposed to new cats infected with FPV, an animal virus, on two occasions. Nothing special was done for the first exposure. The second time an additional dose was given them just after exposure. After these two exposures, which were several months apart, all of the old cats tested positive for FPV. However, none of the cats came down with any symptoms and are in excellent health at the time of this writing.

From this it should be obvious that this composition is also effective against animal viruses.

### Example 2

In a different embodiment of this invention another suitable composition for a product according to the present invention is shown in the following table:

| | Weight % | Weight % | Human |
|---|---|---|---|
| Carbohydrate | (range) | (tested) | (mg) |
| D-Galactose | 0.1 to 50 | 7.2 | 25 |
| L-Fucose | 0.1 to 90 | 14.4 | 50 |
| D-Mannose | 1.0 to 70 | 31.6 | 110 |
| D-Xylose | 0.1 to 70 | 10.8 | 38 |
| Glucosamine HCL | 1.0 to 50 | 10.8 | 38 |
| Sialic Acid | 0.1 to 50 | 10.8 | 38 |
| N-Acetyl-Galactosamine | 0.1 to 90 | 14.4 | 50 |

In this composition the ingredients Glucosamine HCL and D-Mannose are optional and preferred. In this composition D-Galactose is not considered to be optional as this composition is intended for animals as well as humans. This composition was packaged in a size #1 capsule and taken once a week.

This composition is considered a wellness mixture. It was designed to reduce the probability of infections, like colds, while taking the capsules. These ingredients provide Anti-Viral, Anti-Fungal and provide defense against Gram Negative Bacteria. The initial starting composition was derived from the findings of many independent researchers each working with just one of the essential sugars or their derivatives. This combination was initially derived from the findings of independent researchers.

The composition was then refined from the results of human and animal tests to the mixture shown.

In recent studies this composition was also found to be extremely effective at mitigating the effects of handover due to over consumption of alcohol by taking a single dose early in the morning.

### Example 3

In a different embodiment of this invention another suitable composition for a product according to the present invention is shown in the following table:

| | Weight % | Weight % | Human |
|---|---|---|---|
| Carbohydrate | (range) | (tested) | (mg) |
| D-Mannose | 1.0 to 40 | 28.8 | 100 |
| Glucosamine Sulfate | 0.1 to 30 | 20.8 | 72 |
| D-Xylose | 0.1 to 70 | 28.8 | 100 |
| Sialic Acid | 0.1 to 50 | 21.6 | 75 |

In this composition the ingredient D-Mannose is optional and preferred. This composition was packaged in a size #1 capsule and taken twice a day when a viral infection is eminent. This composition is good for both humans and animals.

This composition is considered a cold pill mix. It is intended to reduce the effects of viral infections, like colds, and speed recovery. These ingredients provide Anti-Viral, Anti-Fungal and defense against Gram Negative Bacteria the same as in the wellness mixture, shown in Example 2, except here the amounts have been raised to achieve the maximum beneficial effect.

When taken after the onset of a cold dramatic relief is felt within four hours of taking this composition. The two doses should be taken 12 hours apart and not within two hours of a meal. Generally 10 PM and 10 AM seemed to work best for the test subjects. In some cases just taking a single dose at bedtime was sufficient to completely end all of the symptoms by morning.

### Example 4

In a different embodiment of this invention another suitable composition for a product according to the present invention is shown in the following table:

| | Weight % | Weight % | Human |
|---|---|---|---|
| Carbohydrate | (range) | (tested) | (mg) |
| L-Fucose | 0.1 to 80 | 21.6 | 75 |
| Glucosamine HCL | 1.0 to 50 | 28.0 | 97 |
| Sialic Acid | 0.1 to 50 | 21.6 | 75 |
| N-Acetyl-Galactosamine | 0.1 to 90 | 28.8 | 100 |

In this combination the ingredient Glucosamine HCL is optional and preferred. This composition was packaged in a size #1 capsule and taken once a day. It is good for both humans and animals.

This composition is considered an anti-aging plus learning and memory enhancer mixture. It is intended to reduce the effects of cell aging and enhance memory and learning ability. This combination will not reverse any current level effects already present but should help to reduce the rate at which the cells age progressively. This combination was derived from the findings of independent researchers. Most of the research relating to aging using the essential sugars was done with animals.

### Example 5

In a different embodiment of this invention another suitable composition for a product according to the present invention is shown in the following table:

| | Weight % | Weight % | Human |
|---|---|---|---|
| Carbohydrate | (range) | (tested) | (mg) |
| L-Fucose | 0.1 to 90 | 28.8 | 100 |
| Glucosamine HCL | 1.0 to 50 | 42.4 | 147 |
| Sialic Acid | 0.1 to 60 | 28.8 | 100 |
| N-Acetyl-Galactosamine | 0.0 to 1 | 0.0 | 0 |

In this composition the ingredient Glucosamine HCL is optional and preferred. This composition was packaged in a size #1 capsule and taken once a day. It is good for both humans and animals. While this composition uses the same ingredients as Example 7 the amount of N-Acetyl-Galactosamine has been reduced to zero for this use.

This composition is considered a learning enhancer mixture. It is intended to increase the ability of one to learn new tasks and to improve memory. This composition has application in learning disorder diseases. This combination was derived from the findings of independent researchers. Most of the research relating to learning ability using the essential sugars was done with animals.

The initial starting composition was derived from the findings of many independent researchers each working with just one of the essential sugars or their derivatives. The composition was then refined from the results of human and animal tests to the mixture shown. Most of the research relating to learning ability using the essential sugars was done with animals.

### Example 6

In a different embodiment of this invention another suitable composition for a product according to the present invention is shown in the following table:

| | Weight % | Weight % | Human |
|---|---|---|---|
| Carbohydrate | (range) | (tested) | (g) |
| Honey | 1.0 to 99 | 91.6 | 11.0 |
| D-Mannose | 0.1 to 50 | 4.2 | 0.5 |
| Glucosamine HCL | 0.1 to 50 | 4.2 | 0.5 |

In this composition the ingredient Glucosamine HCL is optional and preferred. This composition was administered in a liquid form. This composition is designed as a treatment for GERD. The honey used was Clover Honey, made by bees, that was filtered and processed at a temperature under 140 degrees F. During our testing we found honey that was not filtered or that was processed at a different temperature did not work reliably at treating GERD.

In 2006 a test was started using this composition and is still ongoing today. During the first two weeks a 12 gram dose of the composition was taken twice a day for the first two weeks. The dose should not be taken within two hours of meal times or when the stomach is full. Sugars that are consumed during meals are considered food by the body and are metabolized as such, while sugars taken in-between meals are treated differently by the body.

After the first two weeks a 12 gram dose was taken at bedtime everyday. The dose is taken at least two hours after finishing the last meal of the day and prior to going to bed. This composition required three days before it became effective. By ‘effective’ it is meant that for the first three days that there was no reduction in the GERD condition. However, after the third day the GERD symptoms completely went away in all individuals in the test group. This composition was also tested on type 2 diabetics and it did not noticeably affect their blood glucose level and they did not require any insulin to maintain their proper glucose levels.

While the GERD symptoms at night stopped after the third day of taking this composition this composition's effectiveness became more pronounced after taking it consecutively for a period greater than 30 days. After this period this composition could be missed one evening and the GERD symptoms did not come back that night. The next evening a single dose of this composition would be taken as usual and the GERD symptoms did not return.

This composition is a combinations of sugars which are all either digested or excreted from the body leaving no residues behind as is left by all PPI medications. So far no adverse effects or allergies to this composition were noted in the test group over the last year of testing.

During the year long test there was a single incident where non-acid reflux was present for a period of one day. This deviation was found to be caused by the effects of a food poisoning episode. After the food poisoning episode was over so was the deviant reflux problem.

### Example 7

In a different embodiment of this invention another suitable composition for a product according to the present invention is shown in the following table:

| | Weight % | Weight % | Human |
|---|---|---|---|
| Carbohydrate | (range) | (tested) | (g) |
| Honey | 1.0 to 99 | 91.6 | 9.0 |
| D-Mannose | 0.1 to 50 | 4.2 | 3.0 |

This composition was administered in a paste form. This composition is designed as a another effective treatment for GERD.

It should be noted that this invention does not require any type of apples, or any other fruits, or any type of vinegars, or anti-acids, or any kind of calcium carbonate, or coral calcium, molasses, whole honey, wild flower honey, desert honey, natural honey, artificial honey, unfiltered honey, or raw honey. This invention uses a specially prepared type of bee honey whose composition was discovered during the first year of testing. This bee honey is filtered clover honey, from Montana or North Dakota, that has been processed between 100 degrees F. and 140 degrees F. and then is mixed with a composition of one or more essential sugars.

As the honey used in this invention has been filtered and heat pasteurized the risk of it containing ‘clostridium botulinum spores’ is extremely low. Clostridium botulinum spores are the reason why honey and other sweeteners are not recommended for infants less than a year old. According to the Center for Disease Control 90% of all the food borne clostridium botulinum cases reported in the United States since 1999 came from California, Utah and southeastern Pennsylvania. Imported honey from outside of the United States has also been found to contain clostridium botulinum spores.

These GERD compositions have been shown to be as effective at treating GERD symptoms as any of the leading PPI medications. In addition, these GERD compositions do not have the risks and side effects associated with all PPI medications. These GERD compositions have also been shown to have 1/10 the occurrence of non-acid reflux that all of the leading PPI products exhibit.

The GERD composition can be administered in many ways. It can be made into a jelly and spread on a medium such as a small slice of bread and eaten, it can be added to coffee, tea or other liquid drinks and drunk. It can be made as a liquid and added as a topping to another food substance such as a small bowl of cereal. It can be made very thick like a paste or made into a very thin viscous like liquid. A convenient way to administer the GERD composition is to make it into a thin paste and put in a squeeze tube, like toothpaste. The contents of the tube can then be squeezed out, dispensed, into a spoon for measuring or into the mouth directly.

It can be seen that this invention has a large number of possible beneficial compositions utilizing just one, or any combination, of the seven essential sugars specialized for a specific target. Just because a combination is not specifically set out herein should not limit the scope of this invention. It has been sufficiently shown that there are numerous compositions available with useful purposes by the detailed examples set out herein.

Additionally, the weighting of a composition ingredient will specify a new composition for a new target use even though the ingredients for two different target uses are the very same, refer to Examples 7 and 9. By changing the amounts of an ingredient its effects on cell absorption will change and by increasing or reducing an ingredient within a cell it will turn on or off different genes which will alter the body's response; The Geno Type Diet by Dr. Peter J. D'Adamo, Broadway Books, 2007, ISBN 978-0-7679-2524-2.

In summary, this invention pertains to the field of dietary supplements and nutritional support for promotion and maintenance of optimal good health. More specifically, the invention relates to compositions of seven essential sugars/carbohydrates as dietary supplements that are essential for a mammal's optimal health and functionality and to a new and healthier alternative to PPI medications for the treatment of GERD.

This invention will correct the problem caused by modern diets consisting of highly refined foods, from which many essential ingredients have been eliminated during processing, specifically the seven essential sugars needed for a properly functioning mammal. It will also cure the problem inherent in most of the glyconutrients available today that contain only trace amounts of these essential sugars while containing large amounts of inactive ingredients that can and do cause numerous allergic reactions with no benefit realized.

The above is a detailed description of particular embodiments of the invention. Those of skill in the art should, in light of the present disclosure, appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

Since other modifications and changes varied to fit particular operating requirements and environments will be apparent to those skilled in the art, the invention is not considered limited to the examples chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A composition of at least one, or more, carbohydrates selected from the following group: mannose, n-acetylneuraminic acid, n-acetylgalactosamine, xylose, glucosamine HCL, glucosamine sulfate;
and
honey.

2. The compositions of carbohydrates in accordance with claim 1, further comprising one, or more, form selected from: spreads, jelly, paste.

3. The compositions of carbohydrates in accordance with claim 1, further comprising one, or more, selected from: sweeteners, spices, filler ingredients, flowing agents, lubricants, binders, emulsifiers, fragrances.

4. The compositions of carbohydrates in accordance with claim 1, further comprising one, or more, non-toxic vitamins or minerals.

5. The compositions of carbohydrates in accordance with claim 1, further comprising one, or more, non-toxic herbal, fungal, plant or animal derived agents.

6. The compositions of carbohydrates in accordance with claim 1, used as a dietary supplement.

7. The compositions of carbohydrates in accordance with claim 1, administered by any of the following methods: ingested powder, capsule, caplet, tablet, liquid, lotion, paste, spread, jelly, hypodermic injection, skin patch, subcutaneous injection, suppository, inhalation, nasal spray, suave, squeeze tube.

8. A method for the treatment of gastroesophageal reflux disease, GERD, the method comprising administering a therapeutically effective amount of a compound made from honey and a mixture of the seven essential sugars to a patient suffering from gastroesophageal reflux disease, GERD.

9. The method according to claim 8 wherein the daily dose is within the range of 0.1 gram to 500 grams.
